(19) United States
(12) Patent Application Publication
(10) Pub. No.: US 2022/0409423 A1
(43) Pub. Date: Dec. 29, 2022

(54) MENSTRUAL CUP

(71) Applicant: Pramod PRIYA RANJAN, Ranchi (IN)

(72) Inventors: Pramod PRIYA RANJAN, Ranchi (IN); Nachiket THAKUR, Pune (IN); Alomi PARIKH, Mumbai (IN); Shreya YENGUL, Pune (IN); Dhimant SURYAKANT PANCHAL, Pune, Maharashtra (IN)

(21) Appl. No.: 17/777,201

(22) PCT Filed: Nov. 24, 2020
(86) PCT No.: PCT/IN2020/050978

§ 371 Date: May 16, 2022

(30) Foreign Application Priority Data
Nov. 25, 2019 (IN) 201931048034

(51) Int. Cl. A61F 5/455

(52) U.S. Cl. CPC A61F 5/4553

(57) ABSTRACT

Disclosed is a user-centered menstrual cup (100) that can be placed and removed from the vaginal canal of the human body for menstrual hygiene. The menstrual cup (100) comprises a chamber (10), a rim (20), a band (30), an inward curvy plane face (40) at front side and a flat plane face (50) at back side, ribs (extrusions) (60), grooves (70) and a stem (80). The menstrual cup (100) is user-centered as it is much easier to insert in and remove from the vagina, spill resistant, ergonomic and intuitive. The menstrual cup (100) helps users to ‘comfortably switch’ to sustainable, economical and healthy menstruation experience. The unique design leading to invention helps to reduce the mental load and concerns of users which helps them to easily and comfortably switch to it as well as adapt quickly for regular usage.

### FIELD OF INVENTION

The present invention relates to menstrual hygiene products and more particularly, to a user-centered menstrual cup to be used in menstruation cycles that can be placed in vagina to collect the menstrual blood.

### BACKGROUND OF THE INVENTION

Currently single time use typical sanitary napkins are the once most commonly used as a menstrual hygiene product. To these next are tampons. The sanitary napkins and tampons produce lot of waste which end up in landfills or chock drainage. These are usually collected by cleaning workers who then get exposed to unhygienic scenario. These products may act as breeding ground for the infectious microorganisms. The sanitary napkins and tampons also take about hundreds of years to decompose since they consist of plastic component at some level and hence hazardous to environment and health. Since, the sanitary napkins and tampons are single time use products, these are economically costly for a large section of potential users as they need to be repeatedly purchased. Also, all these absorbs blood and do have chemicals in it which does sometimes lead to rashes and various hygiene issues. Also, the tampons are capable of absorbing the fluid, but when fallen apart it may leave fibers inside the vagina which can cause infection. Tampons have very high chances to give TSS (Toxic Shock Syndrome) when placed in Vagina as when placed for more duration than that of their absorbing capacity.

In prior art, reusable sanitary napkins are also available, which can be reused till couple of months, but if not cleaned, dried and handled properly then can lead to hygienic issues. Also, since such reusable sanitary napkin absorbs the blood, it may be difficult, uncomfortable and irritating for users to clean it. The same problem is caused with period panties.

The prior art also discloses biodegradable sanitary napkins and tampons, which basically means are environment friendly, but they are available at a higher price hence not economically viable for all, being a single time usage product and need to be purchased regularly.

All such products like sanitary napkins which absorb the blood might give odor which is not a pleasant experience. If sanitary napkins are not properly placed, they may cause seepage/leakage of blood, resulting in mental, social and physical discomfort. Since these products, especially tampons absorb all the liquid/fluid in the vagina with blood, leads to making the vagina dry and also changing the PH value which is not healthy.

A replacement for the above mentioned menstrual sanitary products that can resolve most of the issues is a menstrual cup. Though the menstrual cup is sustainable environment friendly and economical since it can be reused for years and hygienic since it collects the blood and is made medical grade silicone, but in many cases it is uncomfortable to use. Mainly users mentally as well as physically feel discomfort in inserting it in and removing from vagina. Other than this, there are concerns regarding proper positioning, sealing, spillage, leakage, ergonomics and others. The shape and size of the vagina varies within the females depending on numerous factors and hence different sizes or shape of the menstrual cup is required.

Difficulties associated with the normal/standard commonly available menstrual cup are listed below:
1. The menstrual cup is difficult to insert in vagina, due to its large area at the top opening.
2. The menstrual cup is difficult to insert in the vagina, as when folded to insert, the forces of a top rim is projected outward (as the entire rim is on the outer side), which leads to making the opening area larger.
3. While insertion the user has to hold the menstrual cup near the rim at the top after folding to lesser the opening area, which leads to the user putting the finger deep inside the vagina to place the cup making the experience uncomfortable and unhygienic.
4. The menstrual cup has no effective intuitiveness which can guide or suggest the user of ways to insert it in or remove from vagina or in general usage.
5. The menstrual cup has no indication or measure of how to treat it, such as to be assured that the cup has properly popped and got sealed after placing it in the vagina. Without no indication or assurance, the user will be in fear of possible leakage of blood or slipping of the menstrual cup.
6. The menstrual cup doesn't provide instant feedback of breaking the seal when pressed/pinched from side while removal and also the breaking of seal is not very effective. Due to this, there is also a possibility of giving extra pressure from fingers or thumb which can lead to spillage. In the menstrual cup, the profile of side walls is convex like curve outward, so when pinched/pressed to break the seal, the wall first tries to become straight first, then concave like a curve inside which is like opposite curve to what was outside and then beyond a point when pressure is provide the seal gets break. The user is not able to see the process of breaking of the seal and hence due to delayed feedback and inefficient breaking of the seal, the user might feel discomfort as well as mental load.
7. To break the seal of the menstrual cup with the vagina wall for removal, the user needs to pinch the side(s) of the cup near the rim at the top for effective breakage for which user have to put the finger deeper inside which is experientially uncomfortable and increases the unhygienic aspect.
8. As the menstrual cup visually also looks large after folding also, for the user to insert it in vagina through their vulva, and the menstrual cup having very low intuitiveness, leads to user having a mental load for insertion.
9. As the menstrual cup has no indication of how to remove the cup from the vagina, creates a mental load of user regarding how to remove.
10. The menstrual cup has long stem at the bottom, which people feel-like/perceive it should be just pulled to remove the menstrual cup is a wrong perception. Doing so without breaking the seal first, will create drag and hence pain, as vacuum has not yet released in this case.
11. While removal, if the entire rim area comes out at once, actually create a discomfort and hence one side should come out gradually and then another.
12. The blood can easily spill out of the Cup while removal if not handled properly.
13. The menstrual cups are sometimes not ergonomical and/or comfortable for people with lower cervix.
14. The menstrual cups have no proper indication at the stem that from where it can be cut to reduce the length of the steam such at it can become more comfortable and ergonomical for the user to place the menstrual cup in vagina, particularly for one with lower cervix.

Due to not being a user-centered design, usage of the device may cause single or combination of physical and mental discomfort to the user.

Accordingly, there exists a need to provide a menstrual device that is environment friendly, hygienic, economical as well as comfortable to user by being user-centered or easy to use which can be comfortably switched to and regularly being used during menstrual cycles.

### OBJECTS OF THE INVENTION

An object of the present invention is to design an innovative menstrual cup, such that it is user centered or easy to use for female and transgender, when on their menstrual cycles, such that they can easily adapt and/or switch top menstrual cup, without any discomfort or mental load and regularly use there onwards.

Another object of the present invention is to provide a menstrual device (menstrual cup) that is easy to insert in and easy to remove from the vagina.

Further object of the present invention is to design the menstrual cup to resist the spillage and leakage of blood while removal of the menstrual cup from the vagina.

Yet another object of the present invention is to design the menstrual cup ergonomical such that people with different physics/body/vaginal structure (for example, people with low cervix, etc.) can feel comfortable in usage.

Furthermore object of the present invention is to design the menstrual cup intuitive with better semantics.

Yet another object of the present invention is to provide a cost effective (economical), environment friendly, reusable (sustainable) and hygienic menstrual cup/product.

### SUMMARY OF THE INVENTION

Accordingly, the present invention provides a user-centered menstrual cup comprises a chamber having an open end at top and a closed end at bottom thereof, a rim provided at the open end of the chamber, a band provided below the rim, an inward curved plane face at front side wall and a flat plane face at back side wall, ribs (extrusions) provided inside at the back wall thereof, grooves provided at the bottom end and a stem provided at the close end of the chamber. The chamber mostly has an outward inclined conical shaped side walls. The rim is a ring like structure having a curvy inclination (can be a straight inclination also) for allowing easy insertion in the vagina through vulva and removal of the menstrual cup from the vagina. The rim is extended inside the menstrual cup thereby making the menstrual cup resistant to spillage of the blood while removing from the vagina. The inclination of the rim provides higher height at the back side of the menstrual cup for an extra space for the blood to get hold/support and to resist spillage of the blood while removal from the vagina, and lower height at the front side of the menstrual cup for making the menstrual cup ergonomical and comfortable to be used by the user having lower cervix. The band has a wall with slightly more thickness than the side walls and the line/step between them acts as a maximum capacity indicator of the blood in the menstrual cup. The band has a number of holes configured thereon for allowing the blood to flow out in case the capacity of the menstrual cup is full and to help to break the vacuum when walls/faces are pinched/pressed. The inward curved plane face and the flat plane face are provided for enabling the menstrual cup easy to remove from vagina, wherein when the user pinch/press the faces, an instant pressure is passed at the top to the rim such as the seal gets break for the vacuum to be released and then the menstrual cup gets pulled out easily. The faces at the front and back sides can vary in number and hence can be on any sides in any amount. Also, the form of the faces can vary and be in combination(s), from being flat plane, curvy plane, concave, hybrid of mentioned or others, as well as can vary in size-surface area. The ribs at the inside of the back wall are provided for resisting the spillage of blood during removal of the menstrual cup from the vagina. The ribs can be horizontal, inclined, vertical or curvy in form as well as can be in any number, at the back wall or on other side or completely at all sides (360°) in continuous or irregular manner. The grooves (cut extrusions) are provided for assured popping and sealing after insertion, as when the user pinch/press the groove with a slight twist/rotation attempt after placing the menstrual cup in the vagina, the rims pops up if not popped yet to form the proper seal with vagina wall. The shape of grooves can change like being a flat plane, curve plane, concave, spiral-screw like and others; as well as number of grooves, surface area-size can also change. The stem is an elongated extension projecting from the bottom end to identify the position and orientation of the menstrual cup inside the vagina. The stem has a step due to change in area of cross-section at the most bottom end thereof to mark/guide the user for cutting the extra stem to increase the comfort by shortening the length of the overall stem, in case the cervix is small or if the stem is being uncomfortable like in case hitting the vaginal wall. All the semantical changes make the menstrual cup much more intuitive. The menstrual cup is made of flexible/elastic material such as medical grade silicone.

### BRIEF DESCRIPTION OF THE DRAWINGS

The objects and advantages of the present invention will become apparent when the disclosure is read in conjunction with the following figures, wherein:

FIG. 1 shows a perspective view of a menstrual cup, in accordance with a preferred embodiment of the invention;

FIG. 2 shows a left side view of the menstrual cup of the FIG. 1;

FIG. 3 shows a front view (A), a back view (B), a right side view (C), a top view (D) and a bottom view (E) of the menstrual cup of the FIG. 1;

FIGS. 4 to 12 shows a perspective view (A), a front view (B), a back view (C), a left side view (D), a right side view (E), a top view (F) and a bottom view (G) of the menstrual cup, in accordance with alternative embodiments of the present invention.

### DETAILED DESCRIPTION OF THE INVENTION

The foregoing objects of the present invention are accomplished and the problems and shortcomings associated with the prior art, techniques and approaches are overcome by the present invention as described below in the preferred embodiments.

The present invention provides a menstrual cup for menstrual hygiene. The menstrual cup is user-centered as it is much easier to insert in and remove from vagina, spill resistant, ergonomic and intuitive. The menstrual cup helps users to ‘comfortably switch’ to sustainable, economical and healthy menstruation experience.

This present invention is illustrated with reference to the accompanying drawings, throughout which reference numbers indicate corresponding parts in the various figures.

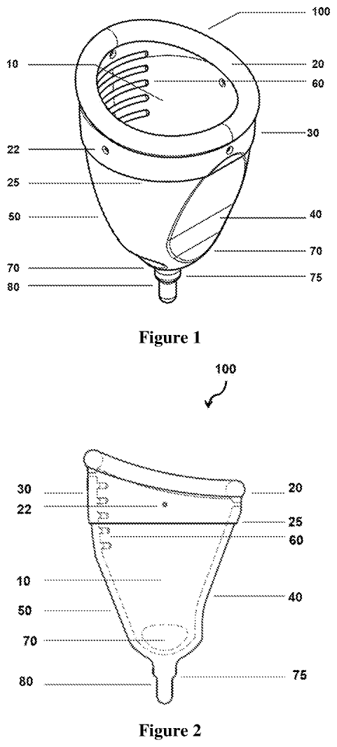

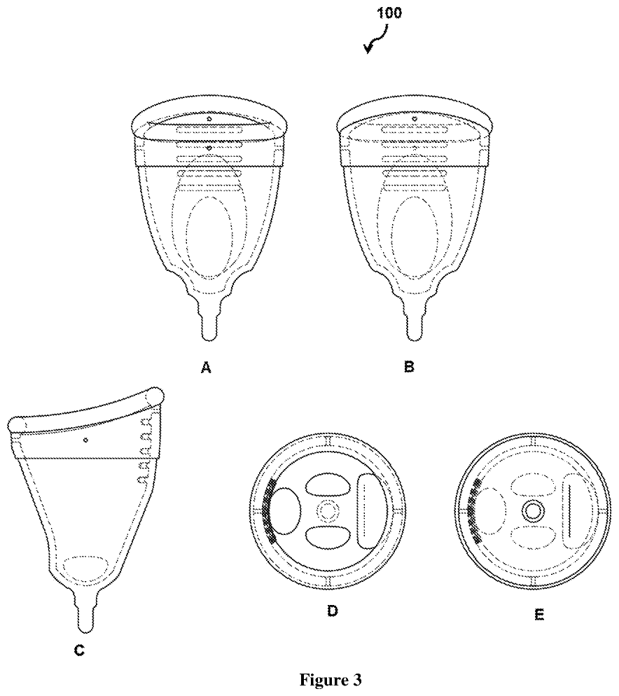

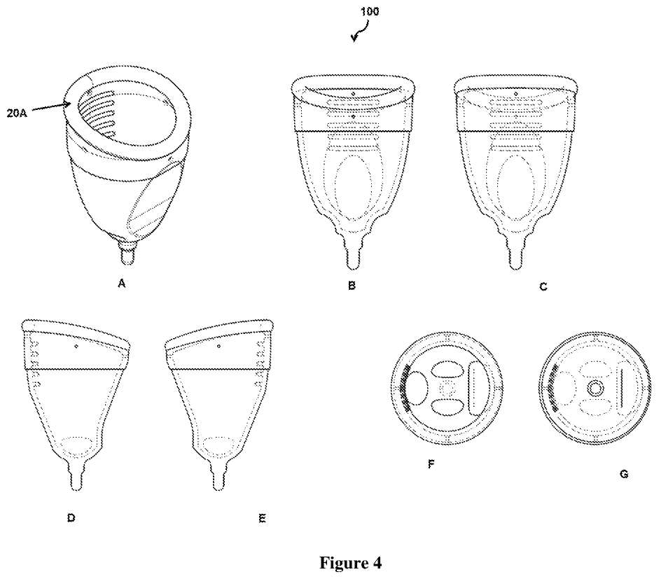

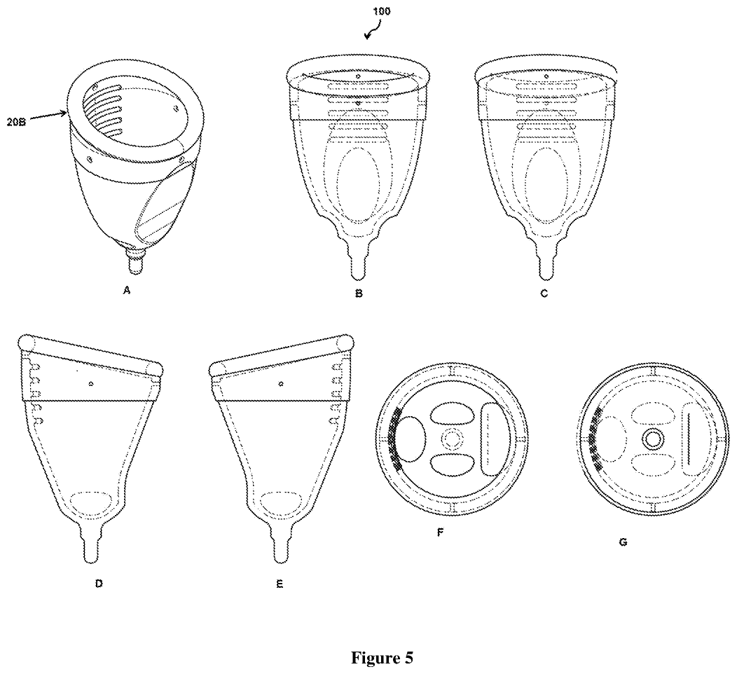

Referring to FIGS. 1 to 3, a menstrual cup (100) for a menstrual hygiene in accordance with the present invention is shown. Specifically, the menstrual cup (100) is user-centered. The menstrual cup (100) comprises a chamber (10), a rim (20), a band (30), an inward curve plane face (40) at front side wall and a flat plane face (50) at back side wall, ribs (extrusions) (60), grooves (70) and a stem (80). Referring to FIGS. 4 to 12, various alternative embodiments of the menstrual cup (100) are illustrated.

The chamber (10) has an open end (not numbered) at top and a closed end (not numbered) at bottom thereof. The chamber (10) mainly has an outward inclined conical-like shape. The cross section of the chamber (10) from top to bottom is partially circular or of any nearby shape, but not limited thereto. The blood is collected inside the chamber (10) during the menstrual cycle.

The rim (20) is provided at the open end of the chamber (10). The rim (20) is a ring like structure that engages to form a seal with the vagina wall and collects the menstrual flow blood into the chamber (10). The rim (20) is inclined. In an embodiment, the inclination of the rim may be a curvy inclination (outward and inward) or a straight inclination. The (curvy) inclination of the rim (20) helps the menstrual cup (100) to get easily inserted in the vagina. To insert the cup, one need to fold it to reduce the top opening area and there are different types of folds which users can use as per their individual convenience. Now, after folding when inserted, the insertion is easy in this menstrual cup (100) as it is gradually getting inserted due to the (curvy) inclined rim (20). The rim side at a larger height is getting inserted first and the rim side a lower height later.

Also, since the rim (20) is having (curvy) inclination, the opening area at the top is much lesser (to that of any standard cup) when folded, which makes it very easy to insert in vagina through vulva as making the insertion gradual, opening area smaller. The rim (20) just straight inclination or inclination with other curvatures also are easy to insert due to similar reasons. Referring to the FIGS. 4 and 5, the menstrual cup (100) is provided with the rim (20) having curvy outward inclination (20A) and straight inclination (20B); respectively. The inclined (curvy) rim (20) also helps in easy and comfortable (pain free) removal of menstrual cup from vagina, as when pulling out, firstly only one side (of lower height side) of the rim (20) of the menstrual cup (100) comes out and then another/opposite side, instead of whole rim coming out as once. The rim (20) is extended inside the menstrual cup (100) which makes the menstrual cup (100) resistant to spillage of blood while removing from the vagina. Also, this rim (20) being (partially or completely) inward helps for easy insertion as when folded, as the opening area is not directing outward as much as it does in normal/standard cup which has rim outward.

Further, due to the inclination at the rim (20), the menstrual cup (100) automatically has a higher height at the back and a lower height at the front. The extra height at the back gives extra space for the blood to get hold/support and hence also resist spillage of blood while removal from the vagina. The smaller height at the front side makes the menstrual cup (100) ergonomical and comfortable to be used by female with lower cervix and in general.

The band (30) is provided below the rim (20). The band (30) has a wall with slightly more thickness than the side walls. The wall thickness difference line (partition-like line) acts as a capacity indicator (25) to show maximum blood collecting capacity of the menstrual cup (100). After couple of cycles, the user gets to understand the amount of blood flow in single usage and can decide the time span of keeping the cup placed in the vagina accordingly. A number of holes (22) are configured at the band that help flow the blood out in case the capacity of the menstrual cup (100) is full and also helps in breaking the vacuum when pinched/pressed.

The menstrual cup (100) has an inward curvy plane face (40) at front side and a flat plane face (50) at back side thereof. The menstrual cup (100) with a permutation and combination of features like said inward curvy plane and flat plane face(s) (40, 50) make the menstrual cup (100) easy to remove from the vagina, as when the user pinch or press the faces (40, 50), an instant pressure is passed at the top to the rim (20) such as the seal gets break for the vacuum to be released and then the menstrual cup (100) can be pulled out easily. Due to the inward curvy and flat plane face(s) (40, 50), the pinching or pressing on the face(s) (40, 50) is effective when also done on the lower side of the cup, and hence the user doesn't need to pinch or press at the top near the rim (20) and can just do near the bottom which is efficient enough to break the seal and hence allowing the user not to letting insert finger(s) or thumb deep inside, also making it comfortable and hygienic.

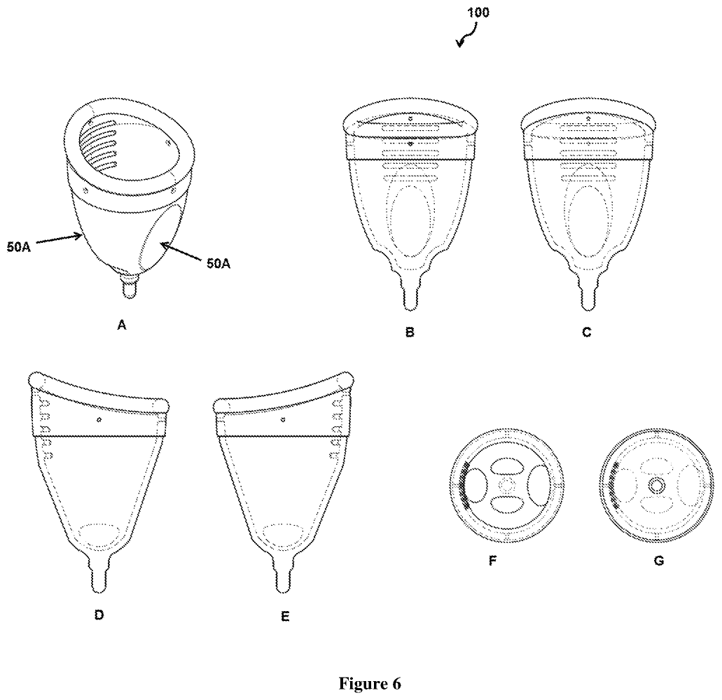

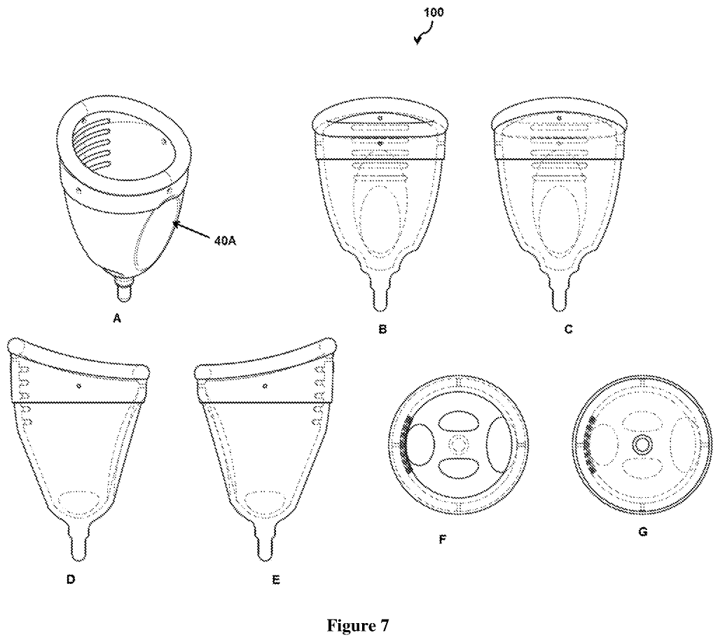

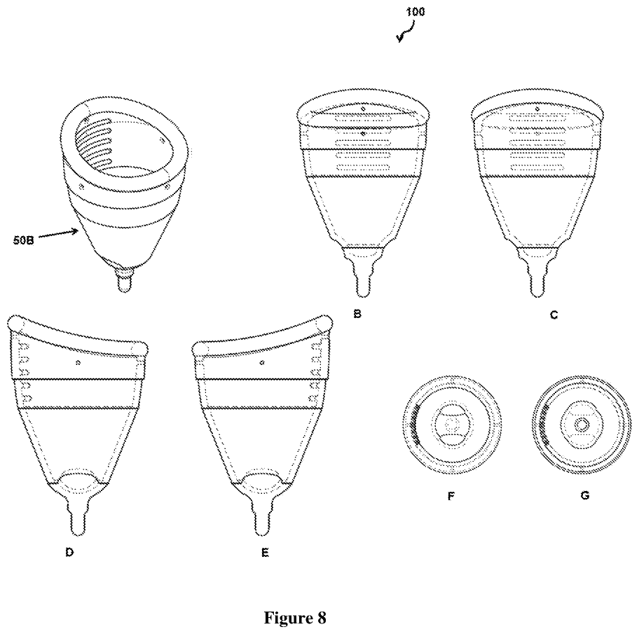

The faces (40, 50) at the front and back sides can vary in number and hence can be on any sides in any amount or throughout. Also, the shapes or form of the faces (40, 50) can vary and can be selected from flat plane, curvy plane, concave, and combinations thereof. Also, the faces (40, 50) can vary in size-surface area. Referring to the FIG. 6, the menstrual cup (100) has flat plane faces (50A) at both sides thereof. Referring to the FIG. 7, the menstrual cup (100) has a concave face (40A) at front side wall thereof.

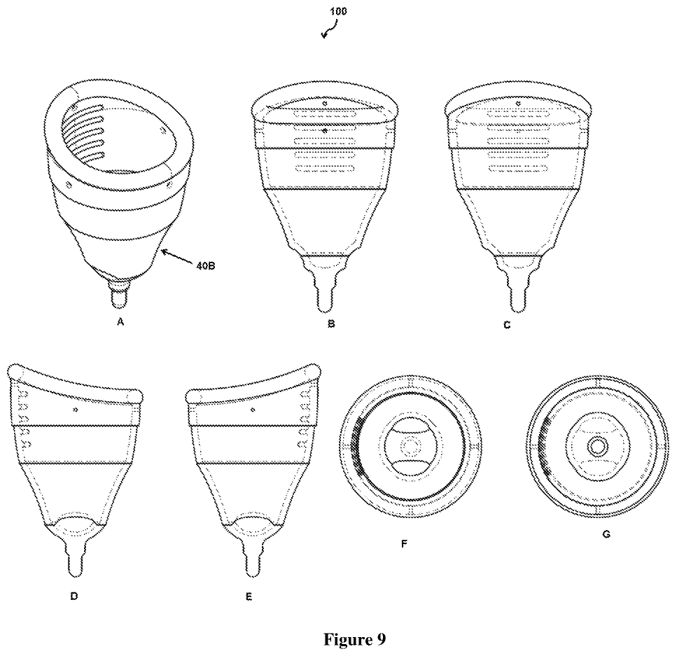

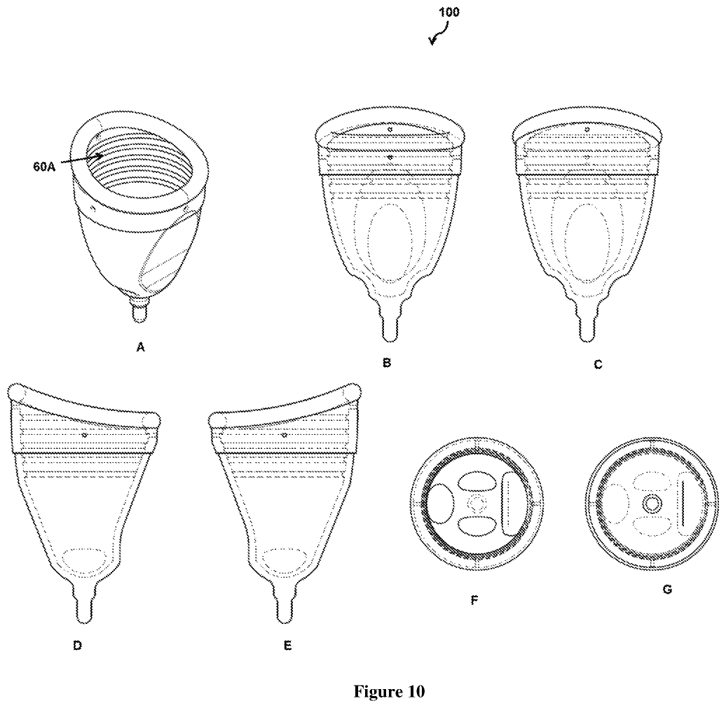

In an embodiment, the faces (40, 50) can also be configured completely (360°) or partially revolving around the menstrual cup (100) to achieve the same objective of easy removal of the menstrual cup (100) from the vagina. Referring to the FIG. 8, the menstrual cup (100) has flat plane faces (50B) at all around (360°) thereof and referring to the FIG. 9, the menstrual cup (100) has concave faces (40B) at all around (360°) thereof.

In the embodiment, the face(s) (40, 50) and other side walls of the chamber, can be of different (variable) wall thickness. In an exemplary embodiment, the face(s) (40, 50) is/are slightly more thicker as compared to other side walls of the chamber (10), thereby making the removal of the menstrual cup (100) from the vagina easy, as instant and efficient feedback can be passed to the rim (20) when user press/pinch the wall to release the vacuum.

In the embodiment, the face(s) (40, 50) and other side walls of the chamber, can be of different (variable) hardness/shore and/or material. In the exemplary embodiment, the face(s) (40, 50) is/are slightly more harder as compared to other side walls of the chamber) and this also leads to making the removal of menstrual cup from vagina easy, as instant and efficient feedback can be passed to the rim (20) when user press/pinch the wall to release the vacuum.

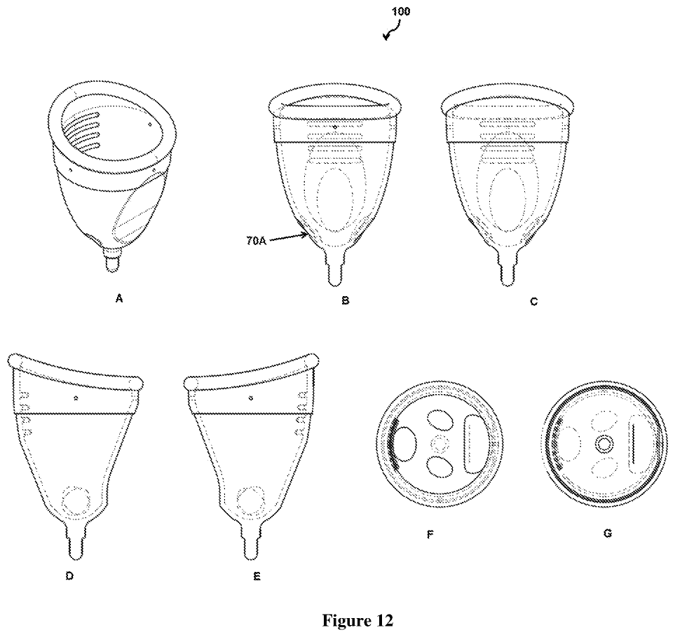

In the embodiment, if there is/are no such face(s) (40, 50), and the selective sides or a portion of any side (for example of front side) of the standard cup is made harder or of a different thickness, then also it leads to making the removal of menstrual cup from vagina easy, as instant and efficient feedback can be passed to the rim (20) when user press/pinch the wall to release the vacuum. Referring to the FIG. 11, the menstrual cup (100) has a curved face (40C) having more thickness and/or different material property with respect to that of the flat plane face (50). The easy removal of the menstrual cup (100) from the vagina can be achieved by increasing the ‘hardness’, which means giving variable ‘hardness’ at the face(s) (or any such part of the cup) where the wall can be pinched/pressed for removal. The variable hardness can be achieved by increasing the thickness of the face(s) (or any such part of the cup) as well as by changing the hardness/shore of the material or by using different types of material or combination of these. In the embodiment, the usual wall thickness of the chamber (10) of the menstrual cup (100) ranges from 1.5 mm to 2 mm. The harder side of the menstrual cup (100) can be 0.3 to 1.5 mm thicker than that of usual/regular wall thickness, that means the harder face (or any part of the cup) can be of 1.8 mm-3.5 mm thick. The usual wall hardness of the chamber (10) of the cup (100) ranges from shore 40-55. The harder face(s) or side of the cup can have 05-20 more shore than usual, that means the harder face (or any such part of the cup) can be of shore 45-70.

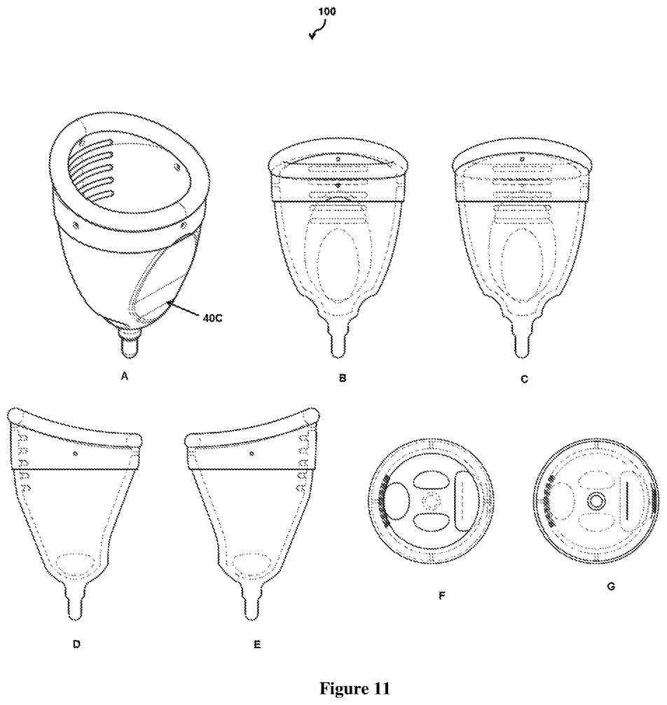

The ribs (60) (extrusions) are provided on inside of the menstrual cup (100) at a back wall thereof. The ribs (60) help in resisting the spillage of the blood while removing from the vagina. The number of the ribs (60) can be varied. In the embodiment, the ribs (60) can be horizontal, inclined, vertical or curvy in form as well as can be in any numbers, just only at the back wall or completely at all sides) (360°) in continuous or irregular manner. Referring to the FIG. 10, the menstrual cup (100) has a plurality number of ribs (60A) at all around (360°) inside thereof.

The grooves (or cut extrusions) (70) are provided to help the user to get assured popping and hence sealing after insertion, when the user pinch/press the groove (70) with a slight twist/rotation, after placing the menstrual cup (100) in the vagina. In the embodiment, the form of grooves (70) can be selected from a flat plane, curve plane, concave, spiral-screw like and combinations thereof. As well as number of grooves (70), surface area-size can also be varied. Referring to the FIG. 12, the menstrual cup (100) have two concave grooves (or cut extrusion) (70A) on the opposite sides at the lower area of the menstrual cup (100) to help the user to hold, slightly press and twist the menstrual cup (100) in a better and easy manner, such as to be sure that the menstrual cup (100) has properly popped and formed the seal with the vagina wall. The axis of these concave grooves (70A) is at an angle (but can be vertical/straight-up as well), but both the concave being at an angle or inclined, make it more efficient to pass the force to form the seal and are intuitive.

The stem (80) is an elongated extension projecting from the bottom end that helps to identify the position and orientation of the menstrual cup (100) inside the vagina. At the most bottom, the stem (80) is provided with a step (75) of different diameter/cross-section area. The step (75) is provided to mark/guide the user that if one feel like cutting the extra stem of a smaller diameter/cross-section then where to cut such as to increase the comfort by shortening the length of overall stem (80), in case the cervix is small or if the stem (80) is being uncomfortable like in case hitting the vaginal wall.

Again referring to FIGS. 1 to 12, fillets are given at the edges of the menstrual cup (100) (where two surfaces meet) can vary in size, keeping the function of the menstrual cup same but to increase the comfort and product durability. The fillets shown in figures are of minimum dimension/size and can be increased such as to achieve more comfort and product durability. In general, a fillet connects two surfaces with a tangent arc in 2D, or creates a rounded transition between the adjacent faces of a 3D solid, such that the edges become soft and continues.

Overall, because of the great semantics of the menstrual cup (100), it is very intuitive to users and helps them to guide in terms of usage and hence reduces mental load. Users easily understand that from which direction it is supposed to be inserted in vagina and where fingers and/or thumbs are supposed to be pinched/pressed among others. The unique design leading to invention helps to reduce the mental load and concerns of users which helps them to easily and comfortably switch to it as well as adapt quickly for regular usage.

In an embodiment, the menstrual cup (100) is made of flexible/elastic material. In a preferred embodiment, the flexible material is medical grade silicone.

### ADVANTAGES OF THE INVENTION

1. The menstrual cup (100) can be easily inserted into the vagina after folding, as this design has inclined (curved or straight) rim (20) which helps in gradual insertion (as compared to normal/standard menstrual cups in which whole rim goes inside at once).
2. The menstrual cup (100) can be easily inserted into the vagina, as when folded the area at opening rim (20) is much lesser due to inclined (curved or straight) rim (as compared to when normal/standard menstrual cups are folded which have horizontal rim).
3. The menstrual cup (100) can be easily removed from the vagina, as it has combination of flat plane and/or inward curvy plane face(s) (50, 40) on sides of the menstrual cup (100) such as when pinched/pressed on it, instant pressure is passed on to the rim and band such that seal which the menstrual cup (100) forms with vaginal wall gets break easily and instantly to release the vacuum formed and hence then the menstrual cup (100) can be pulled out comfortably; also the flat plane and/or inward curvy plane face(s) (50, 40) at sides also helps the user to get instant feedback. (In the normal/standard menstrual cups, the sides are bulging outward or convex and hence when pinched/pressed, then pressure in not instantly passed and is low, also leading to not giving proper or delayed feedback to user and not breaking the seal effectively).
4. The menstrual cup (100) can be easily removed from the vagina, due to inclination of the rim (20), as while removal the side with shorter height will come out first and then the side with larger height (as compared to normal/standard menstrual cups, in which entire rim come out at once if not handled properly and creates discomfort).
5. The menstrual cup (100) resists spillage of blood while removing from the vagina, as it has rim (20) largely inside (as compared to normal/standard menstrual cup which has rim outside).
6. The menstrual cup (100) resists spillage of blood while removing from the vagina, as it has multiple extruded ribs (60) on inside of the menstrual cup (100) (at back wall side).
7. The menstrual cup (100) is easy to insert in the vagina, as the rim (20) in largely inside which helps the menstrual cup (100) to not open outward much when folded (as compared to normal/standard menstrual cups where the rim is opening outward when folded due to rim being in outside).
8. The rim (20) has a variable thickness, which helps to design the menstrual cup (100) in way that it can get folded from particular places for ease while both insertion in and removal from the vagina Similarly, the wall hardness of faces and side wall vary (like the faces or the pressing/pinching side being harder) because of which the menstrual cup (100) is easy to remove as the variable hardness helps to break the seal easily and efficiently when pressed/pinched.
9. The menstrual cup (100) can be hygienically and comfortably inserted in vagina without letting the fingers go deep inside, as the user can just hold near the bottom of the menstrual cup (100) (instead near the rim like in normal/standard menstrual cups) after folding which is enough efficient to place the menstrual cup (100) in the vagina.
10. The menstrual cup (100) can be easily removed from vagina without letting the fingers go deep inside, as pinching/pressing at the bottom of the menstrual cup (100) on flat plane and/or inward curvy plane face(s) (50, 40) is enough to break the seal and pull it out comfortably (as compared to pressing at the top near the rim for the same, like in normal/standard menstrual cups).
11. The menstrual cup (100) has grooves (70) or flat or concave or curvy or similar faces at the bottom of the cup to give a feedback of assured popping and hence proper seal with the vaginal wall, when pressed or slightly twisted after menstrual cup being inserted and placed in the vagina.
12. The menstrual cup (100) has an intuitive design due to great semantics that helps user to understand visually how to handle/interact/use the cup like direction of insertion, folding, etc. and where to place the fingers or thumb, among other conveniences.
13. The menstrual cup (100) can be reused for multiple years and hence is environment friendly/sustainable and economical.
14. The menstrual cup (100) is made of medical grade silicone which means it collects the blood instead of absorbing which makes it much more hygienic and rash-free then other sanitary products.
15. The menstrual cup (100) is placed in the vagina and hence gives an odorless menstrual experience. Also is much friendly for sports and outdoor activities.
16. The menstrual cup (100) holds larger quantity of blood as compared to other sanitary products and hence can be wore for a longer duration.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, to thereby enable others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the scope of the present invention.

1. A menstrual cup comprising:
a chamber having an open end at top and a closed end at bottom thereof, the chamber having an outward inclined conical shaped side walls;
a rim provided at the open end of the chamber, wherein the rim is a ring like structure having an inclination for allowing easy insertion as making the insertion gradual, opening area smaller in the vagina through vulva and removal of the menstrual cup from the vagina as cup coming out gradually and the rim is extended inside the menstrual cup thereby making the menstrual cup resistant to spillage of the blood while removing from the vagina;
a band provided below the rim, the band having,
a wall with slightly more thickness than the lower side walls giving a partition-like line that serves as a maximum capacity indicator of the blood in the menstrual cup, and
a number of holes configured thereon for allowing the blood to flow out in case the capacity of the menstrual cup is full and to break the vacuum when walls/faces are pinched/pressed;
an inward curved plane face at front side wall and a flat plane face at back side wall for enabling the menstrual cup easy to remove from the vagina, wherein when the user pinch/press the faces, an instant pressure is passed at the top to the rim such as the seal gets break for the vacuum to be released and then the menstrual cup gets pulled out easily;
ribs (extrusions) provided inside of the menstrual cup at the back wall for resisting the spillage of blood during removal of the menstrual cup from the vagina;
grooves (cut extrusions) provided at the bottom wall of the menstrual cup, for assured popping and sealing after insertion, when the user pinch/press the groove with a slight twist/rotation attempt after placing the menstrual cup in the vagina; and
a stem provided at the close end of the chamber, wherein the stem is an elongated extension projecting from the bottom end to identify the position and orientation of the menstrual cup inside the vagina.

2. The menstrual cup as claimed in claim 1, wherein the inclination of the rim is selected from a curvy inward inclination, a curvy outward inclination and a straight inclination.

3. The menstrual cup as claimed in claim 1, wherein the inclination at the rim provides higher height at the back side of the menstrual cup for an extra space for the blood to get hold/support and to resist spillage of the blood while removal from the vagina and lower height at the front side of the menstrual cup for making ergonomical and comfortable to be used by the user having lower cervix.

4. The menstrual cup as claimed in claim 1, wherein the faces are configured at any side walls in any amount in shapes selected from flat plane, curvy plane, concave and combinations thereof having varied in size-surface areas.

5. The menstrual cup as claimed in claim 1, wherein the ribs are configured in forms selected from horizontal, inclined, vertical and curvy in any number, at the back wall or on other side or completely at all sides (360°) in continuous or irregular manner.

6. The menstrual cup as claimed in claim 1, wherein the grooves have sizes and shapes selected from flat plane, curve plane, concave, spiral-screw like and combinations thereof, thereby forming a proper seal between the menstrual cup and a wall of the the vagina when pressed/pinched thereon.

7. The menstrual cup as claimed in claim 1, wherein the stem has a step due to change in area of cross-section at the most bottom end thereof to mark/guide the user for cutting the extra stem to increase the comfort by shortening the length of the overall stem, in case the cervix is small or if the stem is being uncomfortable like in case hitting the vaginal wall.

8. The menstrual cup as claimed in claim 1 is made of flexible/elastic material such as medical grade silicone.
